(19) United States
(12) Patent Application Publication
(10) Pub. No.: US 2005/0075282 A1
(43) Pub. Date: Apr. 7, 2005

(54) Materials and methods for inhibiting the development of epilepsy

(76) Inventor: Douglas Coulter, Media, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/956,572

(22) Filed: Oct. 1, 2004

Related U.S. Application Data
(60) Provisional application No. 60/507,679, filed on Oct. 1, 2003.

(51) Int. Cl. A61K 38/12; A61K 31/70; A61K 31/19

(52) U.S. Cl. 514/9; 514/23; 514/557; 514/575; 514/570

(57) ABSTRACT

Provided herein are methods of preventing the onset of epilepsy, or reducing the severity of epilepsy at it's onset, by reducing or eliminating the repression of neurotransmitter receptor gene expression resulting from brain injury.

This application claims priority under 35 U.S.C. 119(e) to U.S. Provisional application 60/507,679 filed Oct. 1, 2003, the disclosure of which is incorporated by reference herein.

Pursuant to 35 U.S.C. §202(c), it is acknowledged that the U.S. Government has certain rights in the invention described, which was made in part with funds from NIH-NINDS grants NS-32403 and NS-38572.

### FIELD OF THE INVENTION

The present invention relates to the fields of neurobiology, medicine, and molecular biology. More specifically, methods are provided for inhibiting or preventing repression of neurotransmitter receptor gene expression upon or during an event causing brain injury, thereby preventing or reducing the severity of subsequent onset of epilepsy.

### BACKGROUND OF THE INVENTION

Several publications and patent documents are cited throughout the specification in order to describe the state of the art to which this invention pertains. Full citations for certain of these references can be found at the end of the specification. Each of these citations is incorporated herein as though set forth in full.

Epilepsy affects an estimated 2.5 million people in the United States and 40 million worldwide. Despite recent advances in treatment, many people with epilepsy still suffer from uncontrolled seizures or from the side effects of treatment. A recent study by the Epilepsy Foundation estimated that the annual financial cost of this disorder is $12.5 billion in the United States alone (White House-Initiated Conference on Epilepsy, 1991.)

Epilepsy may develop as a consequence of brain injury. One prevalent form of such acquired epilepsies is Temporal Lobe Epilepsy (TLE). This prevalence is undoubtedly due to the fragility of the hippocampus, which in turn may be due to the critical role this structure plays in learning and memory. Several characteristic changes occur in the hippocampus of patients and animals with TLE. The relative role these changes may play in the disease process remains an active area of research.

Accompanying the development of epilepsy is a pattern of anatomical changes, including cell loss and sclerosis, circuit rearrangements typified by mossy fiber sprouting, and changes in the expression patterns of ion channels and neurotransmitter receptors in surviving neurons of the epileptic hippocampus (Mouritzen Dam 1980; Babb et al. 1984; de Lanerolle et al. 1989; Sutula et al. 1989; Houser et al. 1990; Babb et al. 1991; Otis et al. 1994; Buhl et al. 1996; Gibbs et al. 1997; Brooks-Kayal et al. 1998; Nusser et al. 1998; Shumate et al. 1998; Brooks-Kayal et al. 1999; Cohen et al. 2003). TLE is a diverse condition, and different patients and animals may have all, some, or only one of these characteristic symptoms. In particular, in data derived from animal models of TLE, it is clear that many of the anatomical changes occurring in the hippocampus are not necessary for the development of epilepsy. In particular, the development of mossy fiber sprouting, and mesial temporal sclerosis may not be necessary. For example, animals which experience SE, but received pharmacological or functional treatments preventing sprouting or cell loss still develop TLE (Longo and Mello, 1998; Andre et al. 2000; Zhang et al. 2002). This does not mean that, when present, these anatomic changes may not be contributory. Rather, it demonstrates that they are neither necessary nor sufficient to explain TLE.

Currently, there is no cure for epilepsy aside from drastic surgical intervention, and physicians are forced to treat symptoms with drugs that often produce undesirable side effects. Therefore a need exists in the art to determine the specific events which lead to the onset of epilepsy, and to target one of more of these events to inhibit or prevent epileptogenesis.

### SUMMARY OF THE INVENTION

Provided herein are methods of preventing the onset of epilepsy, or reducing the severity of epilepsy at it's onset, by reducing or eliminating the repression of neurotransmitter receptor gene expression at, before, or immediately following brain injury.

In particular, reducing or eliminating repression of neurotransmitter receptor gene expression may be by administration of an agent comprising a transcriptional derepressor. The transcriptional derepressor may target gene silencing agents including but not limited to Histone deacetylase (HDAC), DNA methyl transferase, neuron-restrictive silencing element (NRSE), and repressor element-1-silencing transcription factor (REST). The transcriptional derepressor is preferably an HDAC inhibitor. HDAC inhibitors include, but are not limited to valproic acid (VPA), trichostatin A, suberoyl anilide bishydroxamide (SAHA), m-carboxycinnamic acid (CBHA), oxamflatin, trapoxin A, apicidin, topiramate, levetiracetam and sodium butyrate. These agents may be administered alone, in combination with one or more additional transcriptional derepressors, and/or in combination with one or more other anti-convulsant agents.

The transcriptional derepressor may target repression of any neurotransmitter receptor gene, including but not limited to include GABA_{A} receptors (α1-6, β1-3, γ1-3, δ, ε, π), AMPA receptor (GluR1-4), NMDA receptor and kainate receptors (GluR5-7, KA1-2).

In one embodiment, the transcriptional derepressor is administered during a critical window of time, in order to effectively reduce or eliminate neurotransmitter receptor transcriptional repression. This critical window is generally about 7-14 days after brain injury, more preferably 1-3 days after brain injury.

In another embodiment, the transcriptional derepressor is an HDAC inhibitor which is administered to a human in a dose sufficient to achieve clinically effective concentrations in the brain, about 100 to 500 mg/kg/day in an adult, or about 50-400 mg/kg/day in a child. Further information on dosing is provided hereinbelow.

In yet another embodiment, the transcriptional derepressor may be administered by a method including but not limited to oral, intravenous, cutaneous or subcutaneous, nasal, intramuscular, intraperitoneal, and intracerebroventricular routes.

### BRIEF DESCRIPTION OF THE DRAWINGS

FIGS. 1A and 1B are representative traces (FIG. 1A) and a graph (FIG. 1B) showing that neurotransmitter receptor function is repressed by SE.

FIGS. 2A and 2B are representative traces (FIG. 2A) and a graph (FIG. 2B) showing that HDAC inhibitors prevent the repression of GABAR function induced by SE.

FIGS. 3A and 3B are representative tracings (FIG. 3A) and a graph (FIG. 3B) showing that HDAC inhibitors prevent the repression of AMPA receptor function induced by SE.

FIGS. 4A and 4B are representative tracings (FIG. 4A) and a graph (FIG. 4B) showing that HDAC inhibitors alter NMDA receptor function.

FIGS. 5A and 5B are a pair of graphs showing that TSA treatment following SE reduced the subsequent spontaneous seizure frequency.

FIG. 6 is a graph showing that a 1 hour post SE i.p. injection of TSA prevented the repression in neurotransmitter receptor-evoked function observed in 1 week post SE DGCs.

FIG. 7 is a graph quantifying GABA, AMPA, and NMDA receptor responses in dentate granule cells isolated from control animals and animals post-traumatic brain injury. The head injury was inflicted using a standard fluid percussive injury devise, and was equivalent to a severe concussive injury in humans. Neurons have significantly smaller GABA, AMPA, and NMDA currents post head injury, indicating transcriptional repression of neurotransmitter receptors.

### DETAILED DESCRIPTION OF THE INVENTION

The instant invention provides an understanding of the causative mechanisms leading to epileptogenesis, and targets these mechanisms as a means of decreasing the likelihood or severity of epilepsy after brain injury.

Alterations in neurotransmitter receptors of surviving neurons after brain injury play a role in the development of temporal lobe epilepsy. In the hippocampus of chronically epileptic animals, a series of changes in GABAA receptors (GABARs), which are transcriptionally mediated and may contribute to the hyperexcitability responsible for the epileptic condition have been demonstrated (Gibbs et al. 1997; Brooks-Kayal et al. 1998; 1999).

Recently, examination of the properties of neurons after SE-induced hippocampal injury, and before the onset of spontaneous seizures, reveals how these and other alterations in neurotransmitter receptor levels contribute to the disease process itself. This examination reveals a unique set of changes, which can be best described as a broad transcriptional repression in many neuron specific genes, including GABAR subunits (Rikhter et al. 1999; Hsu et al. 2001; Hsu and Coulter, 2002). This transcriptional repression is evident within 24 hrs following SE, persists for 7-10 days, and then resolves by 2 weeks post-SE, usually prior to the onset of spontaneous seizures. Such repression is further evidenced by analysis of mRNA expression profiles in individual dentate granule cells (DGCs), and in patch clamp recording studies which reveal a decrease in the number of functional GABARs in the membrane of these cells.

The discovery disclosed herein demonstrates that this early event in epileptogenesis plays a pivotal role in the subsequent disease process, and that pharmacological interventions to block this initial transcriptional repression event are a viable therapeutic strategy to combat the subsequent development of epilepsy in the injured brain.

The pharmacological agents utilized herein are useful to prevent or inhibit transcriptional repression of neurotransmitter receptors. Therapeutic agents targeting transcriptional repression have been under development in the cancer field for a number of years. Inappropriate transcriptional repression is a hallmark of malignant cells. This is frequently caused by inappropriate cytosine hypermethylation and hypoacetylation of histones in chromatin, leading to hypercondensed chromatin structure in promoter regions of various genes. This renders them inaccessible to transcriptional machinery (reviewed in Jones and Baylin, 2002). Several classes of therapeutic agents have been developed targeting the enzymes mediating these chromatin changes, including DNA methyltransferase (Szyf, 2001; Goffin and Eisenhauer, 2002; Karpf and Jones, 2002), and histone deacetylase (HDAC; Johnstone, 2002; Thiagalingam et al. 2003). HDAC activity is an important component of DNA methylation-induced silencing of genes. DNA methylation occurs at CpG dinucleotides, which are particularly enriched in promoter regions of many genes, where they form CpG islands. HDACs can be directly recruited by DNA methyltransferases. In addition, methylated DNA binds methyl-CpG binding proteins, which also recruit HDACs. The combination of methylation and histone deacetylation effectively silences targeted genes. However, in the normal (non-cancerous) genome, DNA methylation is primarily utilized to silence genes in a very prolonged way, such as during development, since DNA methylation is an irreversible reaction. During development, demethylation of silenced genes is hypothesized to occur only via DNA repair enzymes, which remove and replace methylated cytosines (reviewed in Szyf, 2001). The fact that cancer is associated with inappropriate gene silencing, DNA methylation, and histone deacetylation has driven the development of novel therapeutic agents targeting these processes (Szyf, 2001; Johnstone, 2002; Jones and Baylin, 2002).

In addition to working in concert with DNA methylation in long-term gene silencing, HDACs are also involved in dynamic, shorter term regulation of a number of genes, independent of DNA methyltransferase activity. Acetylation of core nucleosome proteins is a reversible process, and is thought to play a major role in transcriptional regulation (cf. Li et al. 2002). In particular, HDACs are critically involved in repression of neuronal genes in non-neuronal cells. One shared regulatory region within the promoters of many neuronal genes is the neuron-restrictive silencing element (NRSE), also known as repressor element-1 (RE1). This motif contains a 21 base sequence, which binds RE1-silencing transcription factor (REST), a zinc finger transcription factor. Non-neuronal cells express REST, which can bind to the RE1 sequence of NRSE within a promoter, and recruit and bind the corepressors coREST and Sin3A (Huang et al. 1999; Naruse et al. 1999; Ballas et al. 2002). These elements in turn each bind 1-2 copies of the enzyme, histone deacetylase. HDAC then can deacetylate histones within flanking chromosomal regions, which in turn can restrict transcription by condensing chromatin, making it inaccessible to normal transcription factors. In addition, recent evidence has emerged that REST/NRSF may function to silence not only genes containing an NRSE/RE-1 within their promoter, but also neighboring chromosomal regions encoding neuronal genes (Lunyak et al. 2002). This latter phenomenon requires recruitment of a corepressor, CoREST, and involves distinct molecular machinery. This raises the possibility that REST may repress whole arrays of genes in a correlated manner, even though only a subset of these co-repressed genes may contain NRSE elements within their promoters.

Recent evidence has emerged characterizing neuronal expression of REST zinc finger transcription factors (Palm et al. 1998). Neurons express REST, and also exhibit neuron-specific splicing of this transcription factor. In particular, two neuron specific RESTs were described, REST4 and 5, which have 5 zinc finger motifs, compared to the 9 found in full length REST. Full length REST and REST4 were found to be widely expressed in brain, as assessed by in situ hybridization analysis. Expression was evident in both the dentate gyrus and area CA1 of the hippocampus. Following kainic acid-induced SE, expression of REST was rapidly up regulated in hippocampus in a regionally specific manner. REST4 and REST mRNA levels were significantly up regulated 4 hrs following SE in dentate gyrus, while CA1 showed a delay in response, with maximal up regulation 24 hrs following SE. Furthermore, both of these RESTs exhibited transcriptional repression activity when expressed in a Neuro-2A test system, demonstrating their potential effectiveness in neurons. Therefore, it appears that in addition to being a transcriptional silencer in non-neuronal cells, the NRSE/REST system is a transcriptional repressor in neurons, which is regulated in an activity-dependent manner by SE.

I. Definitions

The following definitions are provided to facilitate an understanding of the present invention:

The term “epilepsy” describes a chronic disorder characterized by paroxysomal brain dysfunction due to excessive neuronal discharge, and is usually associated with some alteration of consciousness.

The term “epileptogenesis” describes progressive underlying mechanisms which result in the initial onset of epilepsy following brain injury.

The phrase “status epilepticus (SE)” refers to a single, severe seizure event.

Temporal lobe epilepsy (TLE) is a condition characterized by recurrent unprovoked seizures originating from the medial or lateral temporal lobe. The seizures associated with TLE consist of simple partial seizures without loss of awareness (with or without aura) and complex partial seizures (i.e., with loss of awareness). The individual loses awareness during a complex partial seizure because the seizure spreads to involve both temporal lobes, which causes impairment of memory.

A “seizure” is a change in sensation, awareness, or behavior brought about by a brief electrical disturbance in the brain. Seizures vary from a momentary disruption of the senses, to short periods of unconsciousness or staring spells, to convulsions.

Brain injury refers to damage which occurs to the brain, and which may result in neurological damage, leading to epilepsy. Sources of brain injury which may produce epilepsy include traumatic brain injury such as concussive injuries or penetrating head wounds, brain tumors, alcoholism, Alzheimer's disease, stroke, heart attack, and other conditions that deprive the brain of oxygen, meningitis, AIDS, viral encephalitis, and hydrocephalus.

“Dentate gyrus granule cells” or DGCs provide an in vitro model for studying epilepsy.

A “transcriptional derepressor” is a molecule or agent which antagonizes, inhibits, or prevents transcriptional repression, such as the transcriptional repression of neurotransmitter receptors which occurs during status epilepticus.

HDAC Inhibitor (or histone deacetylase inhibitor) is a molecule, or agent which antagonizes HDAC activity, thus acting as a transcriptional derepressor. Exemplary HDAC inhibitors include valproic acid, sodium butyrate, trichostatin A, CBHA, oxamflatin, trapoxin A, apidcidin, topiramate, levitiracetam and SAHA.

The “Blood Brain Barrier (BBB)” is the specialized system of capillary endothelial cells that protects the brain from harmful substances in the blood stream, while supplying the brain with the required nutrients for proper function. Unlike peripheral capillaries that allow relatively free exchange of substance across/between cells, the BBB strictly limits transport into the brain through both physical (tight junctions) and metabolic (enzymes) barriers.

A “critical window” as used herein is the window of time after a brain injury during which epileptogenesis may be inhibited or prevented, if appropriate treatment is administered. Such a treatment may be administration of a transcriptional derepressor, such as an HDAC inhibitor. The transcriptional derepressor may be administered before brain injury, within 24 hours after brain injury, 24 hours to 7 days after injury, 7-10 days after injury, 24 hours to 2 weeks after injury, 24 hours to two months after injury, or 24 hours to 1 year after injury. Most preferably, the treatment is commenced within 1 to 3 days of brain injury.

The term “subject” or “patient” is used herein to refer to a warm-blooded animal, more preferably a mammal, including, without limitation, non-human animals such as rats, mice, cats, dogs, sheep, horses, cattle, and humans.

An anti-convulsant is any agent which depresses nerve activity in the brain, thereby blocking seizures. Anti-convulsants include but are not limited to Mephobarbital (Mebaral®), Pentobarbital (Nembutal®), Phenobarbital (Luminol®, Solfoton®), Chlorazepate (Tranxene®), Clonazepam (Klonopin®), Diazepam (Valium®), Gabapentin (Neurontin®), Tiagabine (Gabitril®), Ethotoin (Peganone®), Fosphentyoin (Mesantoin®), Phenyloin (Dilantin®), Trimethadione (Tridione®), Lamotrigine (Lamictal®), Ethosuximide (Zarontin®), Methsuximide (Celontin®), Phensuximide (Milontin®), Acetazolamide (Diamox®), Carbamazepine (Carbatrol®, Tegretol®), Felbamate (Felbatol®), Levetiracetam (Keppra®), Oxcarbazepine (Trileptal®), Primidone (Mysoline®), Topiramate (Topamax®), Valproic acid (Depakene®, Depakote®), and Zonisamide (Zonegran®).

II. Methods of Preventing or Reducing the Severity of Epilepsy at the Time of Onset by Administering a Transcriptional Derepressor

The methods of the instant invention are particularly drawn to administering a transcriptional derepressor, such as an HDAC inhibitor to a patient having a brain injury, within 24 hours to 2 weeks of the injury, more preferably within 1 to 3 days of injury, thereby reducing the severity of, or preventing the onset of epilepsy.

During the course of status epilepticus, or other forms of brain injury, it has been determined that neurotransmitter receptor transcriptional repression occurs. This repression results in long term development of epilepsy. The methods provided herein prevent or reduce the severity of epileptogenesis, by preventing or reversing this repression.

The patients which may be treated by the methods of the invention include without limitation, patients who have experienced brain injury as a result of any condition or event including but not limited to trauma, brain tumor, alcoholism, Alzheimer's disease, stroke, heart attack, meningitis, AIDS, viral encephalitis, and hydrocephalus, and any other injury which restricts blood flow or oxygen to the brain. The transcriptional derepressors of the invention include, but are not limited to HDAC inhibitors, such as sodium butyrate, valproic acid, SAHA, and trichostatin A, and may also include CBHA, oxamflatin, and trapoxin A, apidcidin.

At least 4 classes of HDAC inhibitors have been characterized (reviewed in Thiagalingam et al. 2003; de Ruijter et al. 2003). The gold standard agent in mechanistic studies exploring HDAC involvement is trichostatin A, a member of the hydroxamic acid class of HDAC inhibitors. Trichostatin A has an IC₅₀ in the nM affinity range in vitro, but is primarily effective in brain only after ICV administration (e.g. Huang et al. 2002). However, it has recently been shown that IP administration of this agent results in observable CNS effects.

Another agent in the hydroxamic acid class is suberoyl anilide bishydroxamide (SAHA), which works in the μM range. SAHA is currently in clinical trials as an anti-cancer agent, and under active study as a potential therapy in neurodegenerative diseases (Hockly et al. 2003). Butyrate and valproic acid are short chain fatty acids, and belong to a second class of HDAC inhibitors. Both have IC₅₀s in the low mM range (0.4 mM for VPA, and 1.5 mM for butyrate), but these drugs are well tolerated following oral administration in rodents in this dosing range (Loscher, 1999; Egorin et al. 1999). Valproic acid is a widely used, broad spectrum anticonvulsant. Its HDAC inhibitor activity is an unwanted secondary action, independent of its anticonvulsant efficacy, which is responsible for its teratogenicity (Phiel et al. 2001; Gottlicher et al. 2001). Butyrate is currently approved for use in the clinical treatment of cancer, and its effects in ameliorating spinal muscular atrophy (due to degeneration of dorsal horn neurons) are currently under investigation (Chang et al. 2001). Both of these agents are effective in changing brain histone acetylation levels following oral administration (Phiel et al. 2001; Ryu et al. 2003).

The transcriptional derepressors of the invention can be optimized for both the timing of delivery and maximal uptake in, for example, cells of the nervous system or brain. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, modulators, drugs (e.g., anticonvulsants or antibiotics) or hormones.

In preferred embodiments, the pharmaceutical compositions also contain a pharmaceutically acceptable excipient. Such materials should be non-toxic and should not interfere with the efficacy of the active ingredient. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]). The precise nature of the carrier or other material may depend on the route of administration. Administration may be by oral, intravenous, cutaneous or subcutaneous, nasal, intramuscular, intraperitoneal, or intracerebroventricular routes.

The transcriptional derepressors of the invention are preferably administered within a critical window of time, such as before an anticipated brain injury, within 24 hours after brain injury, 24 hours to 3 days after injury, 3-10 days after injury, 24 hours to 2 weeks, 24 hours to 2 months after injury, or 24 hours to 1 year after injury.

One, two, or more different transcriptional derepressors may be administered. If multiple transcriptional derepressors are administered, they may be administered concurrently, alternatively (i.e. one on day one, and a second on day two), or in a staggered fashion (i.e. one is administered for a set time, and then another is administered for a set time.)

The dosage of the transcriptional derepressor may be 50-500 mg/kg/day in adults, and 25-500 mg/kg/day in children, with dosage schedule determined by the relative metabolic rate of the given compound, ranging from once to three times daily.

The EC₅₀ for the following HDAC inhibitors in rats is listed below:
Trichostatin A: EC₅₀ ranges from 50-500 nM. 1 μM was used as the positive control in several studies;
Valproate: EC₅₀ from 0.3-0.7 mM;
Sodium Butyrate: EC₅₀ from 0.2-0.5 mM.

In an exemplary protocol, human subjects will be treated with HDAC inhibitors after initial precipitating injury (IPI) in the following manner:
Trichostatin A (TSA) administration. TSA will be administered intravenously in a dosage ranging from 0.1-0.5 mg/kg as soon as the IPI occurred. Since the latent period for humans is much longer than that observed in rodents, the patient would receive a daily injection for about 1-4 weeks.
Valproic acid administration. Valproic acid (2-propyl-pentanoic acid-sodium salt) (VPA) will be delivered orally if possible or by intravenous injection as soon as the IPI occurred. The antiepileptic dosage for valproic acid is also in the range of EC₅₀ of HDAC inhibition. Therefore, the same dosage having an antiepileptic effect could also be used to inhibit HDAC activity. A period of 2-4 days is required for valproic acid to reach the steady state concentration, while HDAC activity should be inhibited immediately after injury. Accordingly, a double dosage will be administered after IPI for several days. The preferred treatment protocol is below: VPA will be administered at 15-60 mg/kg per day for 1-4 weeks, however a double dosage will be used for the initial 2-4 days of administration. Due to the short elimination half-life, the daily dose provided above divided into 2-3 doses is suggested.

Sodium butyrate administration. Butyrate is approximately twice as potent as VPA. The preferred treatment protocol for human subjects entails administration of butyrate at a dose of 10-30 mg/kg for 1-4 week, however double dosages will be administered during the initial 1-4 days of treatment. Tables 1, 2 and 3 summarize results obtained in previous studies. Entries listed as “lab data” are the result of studies described herein.

TABLE 1
The dosage of Trichostatin A used to inhibit HDAC activity.

| Experiments and Effects | Models | Subjects | Dosage | | Reference # |
|---|---|---|---|---|---|
| Prevent the seizure-induced transcriptional repression | In vivo | Rats | 2 | mg/kg, i.p. | Lab data |
| Prevent SE-induced histone deacetylastion at the GluR2 promoter | In vivo | Rats | 1 | μg-intraventricular | 7 |
| Prevent the seizure-induced transcriptional repression | In vitro | Organotypic | 1 | g/kg/day | Lab data |
| | | culture | | | |
| Prevent SE-induced histone deacetylastion at the GluR2 promoter | In vitro | Brain slices | 300 | nM | 7 |
| HeLa cell nuclear extract-completely block HDACs | In vitro | Cell culture | 1 | μM | 4 |
| HeLa cell nuclear extracts | In vitro | Cell culture | 300 | nM | 9 |
| Enhanced acetylation of histone H4 in 3T3 cells | In vitro | Cell culture | 50 | ng/ml | 1 |
| Enhances levels of acetylated histone H3 protein in aging CCN culture | In vitro | Cell culture | 30 | nM for 14 days | 8 |
| Relieves HDAC-mediated transcriptional repression | In vitro | Cell culture | 100 | nM | 5 |
| Induces accumulation of hyperacetylated histone | In vitro | Cell culture | 100 | nM | 5 |

TABLE 2
The dosage of valproic acid used to inhibit HDAC activity.

| Experiments and Effects | Models | Subjects | Dosage | | Reference # |
|---|---|---|---|---|---|
| Prevent the seizure-induced transcriptional repression | In vivo | Rats | 1 | g/kg/day | Lab data |
| Enhances levels of acetylated histone H3 protein in the cortices | In vivo | Rats | 300 | mg/kg, i.p. | 10 |
| HeLa, F9 teratocarcinoma and Neuro2A Neuroblastoma cells | In vitro | Cell culture | 0.3-0.7 | mM | 5, 9 |
| Enhances histone acetylation in HeLa cell nuclear extract | In vitro | Cell culture | Ki = 0.51 | mM | 4 |
| 293T cells-HDAC assay | In vitro | Cell culture | IC50: 0.7-1 | mM | 6 |
| Human HDAC1 | In vitro | Cell culture | IC50 = 0.4 | mM | 9 |
| HeLa cell nuclear extracts | In vitro | Cell culture | 0.5-5 | mM | 9 |
| Enhances levels of acetylated histone H3 protein in aging CCN culture | In vitro | Cell culture | 0.5 | mM | 8 |
| Relieves HDAC-mediatad transcriptional repression | In vitro | Cell culture | 1 | mM | 5 |
| Induces accumulation of hyperacetylated histone | In vitro | Cell culture | 1 | mM | 5 |

TABLE 3
The dosage of Sodium Butyrate used to inhibit HDAC activity.

| Experiments and Effects | Models | Subjects | Dosage | | Reference # |
|---|---|---|---|---|---|
| 293T cells-HDAC assay | In vitro | Cell culture | IC₅₀: 0.3-0.4 | mM | 6 |
| Induces accumulation of hyperacetylated histone | In vitro | Cell culture | 5 | mM-positive control | 5 |
| Induces acetylated histone H4 in S91 and A375 cells | In vitro | Cell culture | 3 | mM | 3 |
| Inhibition of a purified nuclear histone deacetylase | In vitro | Calf thymus | IC₅₀: 0.25 | mM | 2 |

Full citations for the references can be found at the end of the specification.

The following non-limiting examples are provided to further describe certain embodiments of the invention. They are not intended to limit the invention in any way.

The materials and methods set forth below are provided to facilitate the practice of the present invention.

Animals. The Institutional Animal Care and Use Committee (IACUC) of the Children's Hospital of Philadelphia approved all the protocols used in this study. Male Sprague-Dawley rats (Charles-River, Kingston, Pa.) weighing 180-200 gm at the start of experiments were housed one rat per cage in a 12-hour light-dark cycle with access to food and water ad libitum. In some experiments, drugs were administered in the animal's drinking water (See details in specific experiments).

Valproic acid and butyrate administration. Valproic acid (2-propyl-pentanoic acid-sodium salt) (VPA) was administrated in drinking water (6 g/liter) with addition of sodium saccharin (300 mg/kg) one week prior to pilocarpine-induced status epilepticus (SE). Calculating the daily water consumption of rats, the daily dose administration in this study was 1.0 g/kg VPA. During a nursing period of 2-4 day after status epilepticus, VPA was administrated by s.c. injection, since the animals were too sick to drink sufficient water. VPA was continuously administered to rats for one week after SE. Sodium butyrate was administered in food at 1.5 g/kg/day.

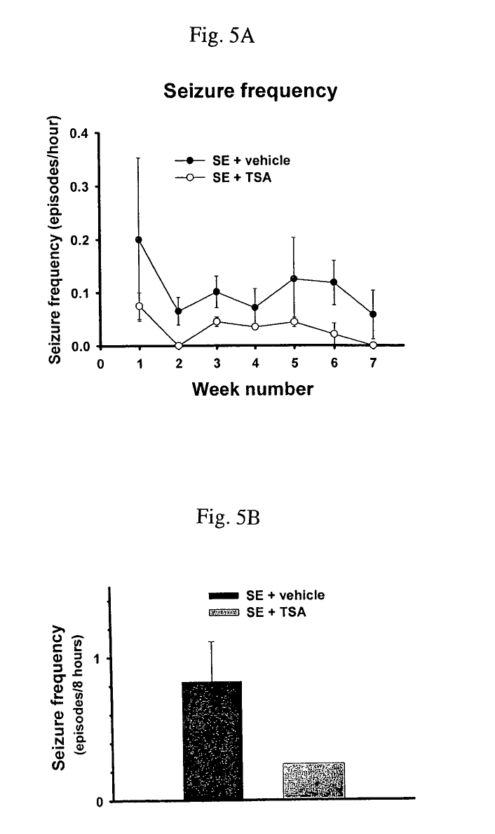

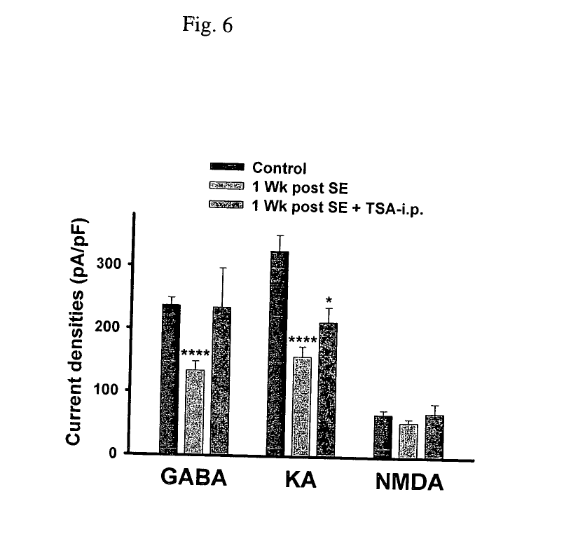

Trichostatin A (TSA) administration. TSA was administered in the lateral ventricle {−0.8 mm A-P; 1.5 LAT; 3.2 DOWN (Paxinos and Watson 1982)} one hour prior to pilocarpine injection to induce SE. The cannula guide had been previously implanted using stereotaxic surgery two weeks prior to experiment to allow animals properly recovery. TSA (5 μl) was delivered via tubing connected to a pump at a concentration of 5 μg/ml (vehicle, 0.9% saline) at a rate of 1 μl/min for 5 minutes through 29-gauge tubing attached to the cannula. Successful delivery of TSA was verified by histological examination of cannula position and a moving mark inside the tubing. In additional studies (FIGS. 5 and 6), TSA was administered I.P., 2, 6, and 24 hrs post SE.

Pilocarpine-induced status epilepticus. Rats were injected with (−) scopolamine methyl nitrate (1 mg/kg body weight, s.c.) 30 min prior to pilocarpine hydrochloride (405 mg/kg body weight, s.c.). Controls received a sub-convulsive dosage of pilocarpine (40 mg/kg, s.c.). After one hour of SE, diazepam was used to stop SE.

Isolation of neurons. Hippocampal dentate granule cells (DGCs) were acutely isolated using a modified published protocol (Smith et al., 1998; Hsu et al., 2003). Brains were harvested in chilled, oxygenated (100%) HEPES medium (155 mM NaCl, 3 mM KCl, 1 mM MgCl₂, 3 mM CaCl₂, 25 mM glucose and 10 mM HEPES, pH, 7.35). The hippocampus was dissected out and cut into 600 μm slices using a McIlwain tissue chopper. The dentate gyrus was further separated and cut into tissue cubes using a surgical blade. These tissue cubes were digested at 30° C. for 30 minutes in a pure oxygen bubbled saline containing (in mM): NaCl 120, KCl 5, MgCl₂ 1, CaCl₂ 1, D-glucose 25, PIPES 20 and protease XXIII 3 mg/ml, pH, 7.2. After digestion, the tissue cubes were rinsed and incubated in enzyme-free medium at a temperature of 8-12° C. to prevent over-digestion and also preserve tissue health. Upon recording, neurons were isolated by trituration in oxygenated HEPES buffer using a series of fire-polished descending diameter glass pipettes. The cell suspension was then plated onto a Petri dish, and allowed to settle 10-15 min before the buffer was replaced with recording medium. DGNs were identified based on their size (10 μm diameter) and distinctive unipolar morphology (round or oval cell body with a single dendritic process).

Electrophysiology. Whole cell voltage clamp techniques were used to record from isolated DGCs. Ligand-gated currents were recorded at room temperature in tetrodotoxin-added (500 nM) HEPES solution. Neurons were voltage-clamped at −20 mV using an intrapipette solution containing (in mM) Trizma phosphate (dibasic) 100, Trizma base 28, EGTA 11, MgCl₂ 2, CaCl₂ 0.5, Mg-ATP 10 and 1 U/μl RNasin, pH 7.35, 290 mOsm/kg H₂O. The ATP regeneration system Tris phosphocreatinine (20 mM) and creatine kinase were added to minimize GABA current rundown. AMPAR and NMDAR-mediated currents were measured at the holding potential of −50 mV to increase the driving force for both receptors. Of additional note, NMDAR-mediated current was recorded in a Mg⁺⁺ free solution. Recording signals were amplified using an Axopatch-1D amplifier and filtered at 5 kHz then recorded using pCLAMP 8.01 software (Axon Instruments, Inc. Foster City, Calif.) for off-line analysis. A solution change time of less of than 3 msec was accomplished via a combination of a solenoid-activated system (Warner Instruments) and step-perfusion device (Warner Instruments). GABA, Kainate, and NMDA (Sigma, St. Louis, Mo.) were applied to the cell for 2 seconds. Current amplitudes and current kinetics were fitted with a single exponential component function (Levenberg-Marquardt nonlinear least-squares algorithm), calculated using Clampfit software (pCLAMP 8.01, Axon Instruments, Inc. Foster City, Calif.).

Analysis of Data. Current density (current normalized to cell capacitance), percentage of current desensitization within 2-second agonist application, time constant for current desensitization, total charge transfer density (calculated by the area under the current trace and further normalized to cell capacitance) were measured in DGCs in both experimental and control groups. Statistical significance of differences between single values for two groups was evaluated using the unpaired Student's t-test or the Mann-Whitney Rank Sum test for groups with unequal variance. One-way ANOVA and Tukey test for post-hoc comparison was used for comparisons of multiple groups. Curves were fitted using the Marquardt-Levenberg nonlinear least-squares algorithm (Clampfit 8.01) to calculate the time constant of desensitization.

Chronic epilepsy monitoring in TSA treatment. Neurotransmitter receptor function was down-regulated after SE induction. This effect lasted for at least 5-7 days, during which time animals had no behavioral seizures (latent period). Animals sequentially developed spontaneous seizures (epilepsy). One hypothesis we proposed is that disrupting the repression in neurotransmitter receptor function seen during the latent period would also interfere with the process of epileptogenesis. To directly test this hypothesis, we examined the effects of TSA injection (I.P.) three times into rats; 2 hr, 6 hr and 24 hr post SE on the subsequent development of epilepsy. Animals were video monitored for 7 weeks post SE, and the frequency and severity of seizures was quantified.

Stereotaxic Surgery Implantation of electrodes and bilateral cannulas (Plastics One Inc) occurs under pentobarbital anesthesia (60 mg/kg, i.p.), with supplemental isofluorane, as necessary. The number of electrodes and the exact sites to be recorded is determined based on the particular experiment. All animals receive bilateral electrodes in the dorsal hippocampus. The Paxinos and Watson Stereotaxic Atlas is used as a guide to determine co-ordinates for the particular sites of interest. Electrodes are constructed of 2 twisted strands of 0.127 mm diameter Nichrome wire, Diamel-insulated, and attached to male Amphenol pins. These components are cut to the appropriate length, surgically implanted and secured to the skull with 6 jeweler's screws. A covering of dental acrylic is then applied.

Acquisition of Seizure Monitoring Data and Analysis All rats remain in their home cage until 10 days following the surgical procedure to allow for recovery. Animals are then connected to the recording leads and continuous monitoring is conducted using a Nicolet 64 channel digital video/EEG system. Animals are connected to the system via mercury commutators, which allow unrestricted movement and facilitate acquisition of long-term recordings. Signals are stored on DVDs for off-line analysis during the protracted duration of continual recordings.

Detection of histone acetylation by Western Immunoblot analysis Hippocampal or neocortical tissue is harvested, nuclear extracts are prepared as described (Huang et al. 2002) and adjusted to 0.4 N H₂SO₄. Precipitated proteins are pelleted, resuspended in 20 mM HEPES, 1 mM EDTA, 1 mM EGTA, and concentrated with Centricon 10 membrane. Samples are separated by SDS-PAGE on 10% gels and immunoblotted with acetyl-histone H3 or H4 antibodies (1:1000; Upstate Biotechnology).

Molecular Biology Methods

Single Cell Antisense RNA (aRNA) Amplification and Expression Profiling on Nylon cDNA Arrays.

The methods are described in Brooks-Kayal et al., 1998; 1999; 2001. Briefly, dedicated stocks of nuclease free chemicals, electrodes, and glassware are used in aRNA amplification experiments, and latex gloves worn at all times. Following a brief (<15 min) physiological recording session, cell contents are collected in a patch electrode via suction, and ejected into an eppendorf tube for subsequent two rounds of aRNA amplification. Electrodes contain 1 U/μl RNasin. First strand cDNA synthesis is accomplished with AMV RT and oligo(dT)T7 primer (5′-AAA CGA CGG CCA GTG AAT TGT AAT ACG ACT CAC TAT AGG CGC T₂₄-3′). Double-stranded cDNA is synthesized with T4 DNA Polymerase and Klenow fragment. Hair-pin loops are cut by S1 Nuclease. Double-stranded template is blunted with T4 DNA Polymerase and Klenow, then drop-dialyzed on filters. Recovered cDNA is used for the first round of aRNA amplification with T7 RNA Polymerase and α-³²P-CTP. The second round of amplification is similar to the first one. First strand cDNA is synthesized with random hexamers and AMV RT. Second strand cDNA is synthesized with T4 DNA Polymerase, Klenow and oligo(dT)T7 primer. Dialysis proceeds as described above. The product of the second round of the aRNA amplification with incorporated α-³²P-CTP serves as a probe to hybridize with reverse Northern slot blots.

Slot-blot preparation. cDNAs used on arrays include GABAA receptor (α1-6, β1-3, γ1-3, δ, ε, π), AMPA (GluR1-4), and kainate receptor (GluR5-7, KA1-2), and housekeeping genes (NFM, NFL, NSE, β-actin, β-tubulin). All cDNAs were sequenced to verify their identity. All plasmid DNAs are linearized by restriction digestion and 1 μg of each is applied on the nylon membrane in a slot-blot apparatus. Each slot blot is hybridized with the radiolabeled aRNA probe from a cell for 40 h at 42° C. in a formamide-containing buffer, washed and exposed to a Phosphorimager screen.

aRNA amplification and expression profiling on glass cDNA microarrays. To prepare target for the glass microarrays, amplification proceeds as described above, except α-³²P-CTP is replaced with Cy3 or Cy5 CTP. cDNAs are linearized by restriction digestion and printed on CMT-GAPS slides using the GM™S 417 Arrayer. Each slide is hybridized with the Cy-labeled target from an individual cell in a formamide-containing buffer using the hybridization chamber ArrayIt™ submerged in a 42° C. waterbath for 20 h at 42° C., washed at RT and spin-dried.

Quantitation and statistical analysis Intensity of the autoradiographic signal on nylon arrays is measured by phosphorimaging (ImageQuant software, Molecular Dynamics). Glass microarrays are scanned in ScanArray 5000 scanner and analyzed using QuantArray software. The value for vector cDNA (pBS SK) is used as a background level for each array. Individual subunits are expressed either relative to summarized receptor signal or NFL signal.

### EXAMPLE 1

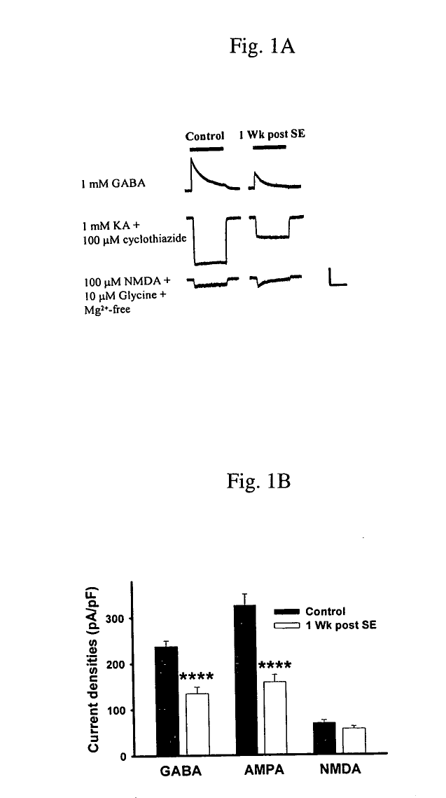

This example describes alterations in neurotransmitter receptor expression and function during the latent period between pilocarpine-induced SE, which injures the brain, and the subsequent onset of recurrent spontaneous seizures. This period usually lasts anywhere from 2-8 weeks, and little is known about neuronal properties during this time. In DGCs, within 24 hrs following SE, these neurons begin to express GABARs with an epileptic phenotype (Brooks-Kayal et al. 1998; Rikhter et al. 2000). Accompanying this transcriptional switch in the production of GABARs is a dramatic decrease in expression of GABAR mRNA, to 10-20% of control levels (expressed as summed signal for 17 different GABAR subunits, relative to expression of housekeeping genes such as NFl and β-tubulin; and quantitated as percentage of control values, FIG. 1; Rikhter et al. 1999; 2000). This latter change persists for 1 week, and resolves to control levels of expression within 2 weeks. In a transgenic mouse engineered with the δ subunit promoter driving expression of lacZ, reporter activity was significantly decreased for 2 weeks, with recovery within 1 month following SE, supporting the concept that this biphasic regulation of GABAR subunit mRNA levels (low in latent period, high in chronically epileptic animals relative to controls) is transcriptionally regulated (Kelly et al. 2000). In addition to the decrease in 17 GABAR subunit mRNAs described above (FIG. 1), expression of other receptor systems was also significantly down regulated in 24 hours and 1 week post SE DGCs. Decreases in expression and function of AMPA (GluR 1-4) and kainate (GluR 5-7, KA 1-2) receptors were evident 1 week post SE (to 10-20% of control values, FIG. 1). By 2 weeks post SE, expression levels of AMPAR and KAR rebounded and were not significantly different from control values.

FIG. 1 herein demonstrates that neurotransmitter receptor function was repressed by SE. FIG. 1A depicts representative traces of DGC responses to GABA (1 mM), KA (1 mM)+cyclothiazide (100 μM; an AMPA receptor desensitization inhibitor) and NMDA (100 μM)+glycine (10 μM) recorded in Mg²⁺-free solution. Current densities for both GABA-A and AMPA, but not NMDA receptors were significantly reduced one-week post SE (1 Wk post SE). (*P<0.05, **P<0.01, ***P<0.001, ****P<0.0001 and scale bar for 500 pA×1 sec). See FIG. 1B.

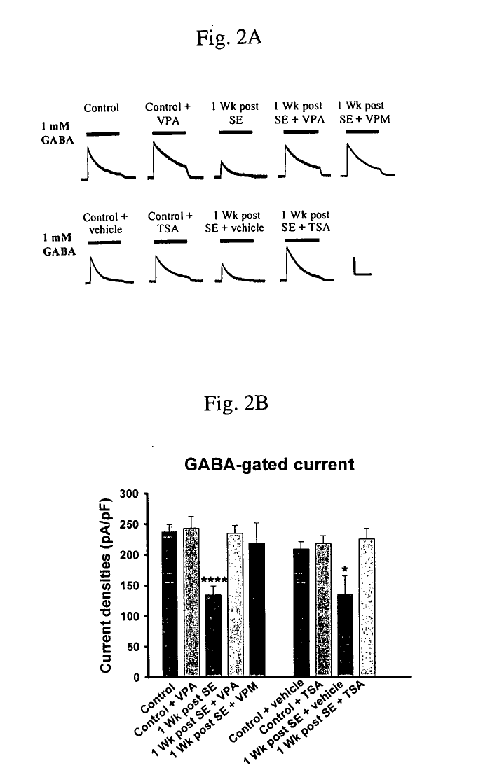

HDAC inhibitors prevent the repression of GABAR function induced by SE as demonstrated in FIG. 2. FIG. 2A depicts representative traces of DGC responses to GABA (1 mM) as indicated. FIG. 2B shows that VPA treatment in control rats does not alter DGC GABAR current densities (Control: 237.5±12 pA/pF; n=67 cells vs. Control+VPA: 243.04±19.25 pA/pF; n=22 cells; p>0.5) indicating that there is no tonic inhibition of HDAC affecting on GABAR efficacy in DGCs. However, VPA treatment prevented the repressed GABAR current densities observed in 1 Wk post SE DGCs. The current density was recovered to the control level. {Control: 237.5±12.0 pA/pF (n=67 cells) vs. 1 Wk post SE+VPA: 234.6±12.7 pA/pF (n=74 cells); p>0.8}. Additionally, valpromide (VPM), the amide of VPA, also prevented the reduced GABAR current densities observed 1 Wk post SE to 218.63±33.2 pA/pF (n=30 cells), a level comparable to its parent compound. In the TSA study, due to the involvement of surgery in TSA administration, a new set of control and experimental rats were assessed. Similar to VPA treatment, TSA treatment in control rats did not alter the GABAR current densities {“Control+vehicle”: 209.1±12.0 pA/pF (n=15 cells) vs. “Control+TSA”: 217.9±12.8 pA/pF (n=36 cells); p>0.4} demonstrating again no tonic inhibition of HDAC on GABAR efficacy. SE-induction also resulted in decreases in GABAR current densities in DGCs recorded 1 Wk post SE. GABAR current densities were significantly reduced to 64.1% of control {“Control+vehicle”: 209.1±12.0 pA/pF (n=15 cells) vs. “1 Wk post SE+vehicle”: 134.1±31.1 pA/pF (n=17 cells); p<0.05}. Similar to the observations made following VPA treatment, TSA treatment also prevented the decreased GABAR current densities observed 1 Wk post SE. The current density was restored to the control level. {“1 Wk post SE+TSA”: 225.6±17.3 pA/pF (n=32 cells) compared to that of “Control+vehicle”; p>0.3}. (*P<0.05, **P<0.01, ***P<0.001, ****P<0.0001 and scale bar for 500 pA×1 sec).

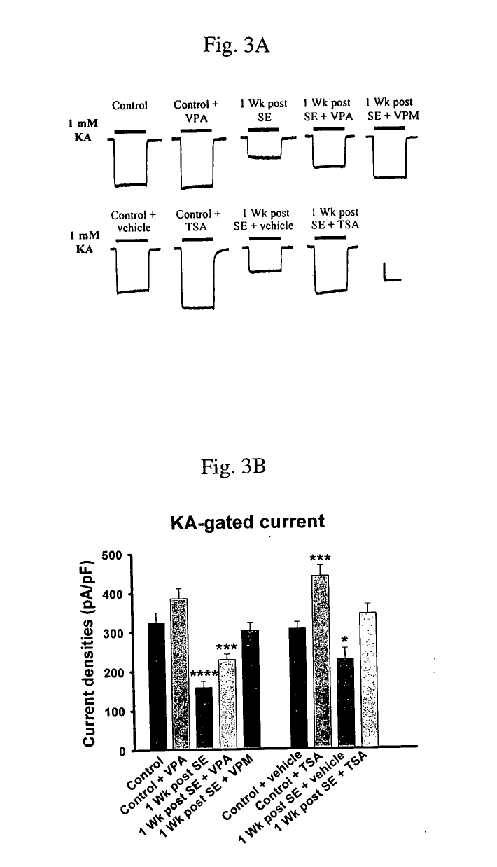

FIG. 3 demonstrates that HDAC inhibitors prevent the repression of AMPA receptor function induced by SE. FIG. 3A depicts representative traces of DGC response to kainate (1 mM) along with cyclothiazide (100 μM), an AMPA receptor-specific desensitization inhibitor. Both HDAC inhibitors displayed the tendency to increase AMPAR current densities in control rats, however, only TSA treatment reached the significance by increasing 43.8% (“Control+vehicle”: 306.3±17.0 pA/pF; n=23 cells vs. “Control+TSA”: 440.6±26.6 pA/pF; n=41 cells) indicating that there is a tonic HDAC inhibitory activity in modulating AMPAR function in DGCs. 1 Wk post SE, AMPAR current densities were reduced to 48.6% of control values {Control: 325.4±24.8 pA/pF (n=38 cells) vs. 1 Wk post SE: 158.1±16.1 pA/pF (n=32 cells)}. This is in contrast the effect observed on GABAR function. VPA treatment was able to partially restore reduced AMPAR function. However, the amide of VPA (VPM) totally prevented the reduced AMPAR current densities observed 1 Wk post SE to 302.5±20.2 pA/pF (n=27 cells). In contrast to VPA treatment, TSA treatment totally prevented the reduced AMPAR current densities in DGCs recorded 1 Wk post SE. In the TSA study, AMPAR current densities were significantly reduced to 74.2% of control {“Control+vehicle”: 306.3±17.0 pA/pF (n=23 cells) vs. “1 Wk post SE+vehicle”: 227.2±28.4 pA/pF (n=18 cells)}. Additionally TSA treatment was able to prevent the decreased AMPAR function observed 1 Wk post SE compared to that of “Control+vehicle” but not the level of “Control+TSA”. (*P<0.05, **P<0.01, ***P<0.001, ****P<0.0001 and scale bar for 500 pA×1 sec).

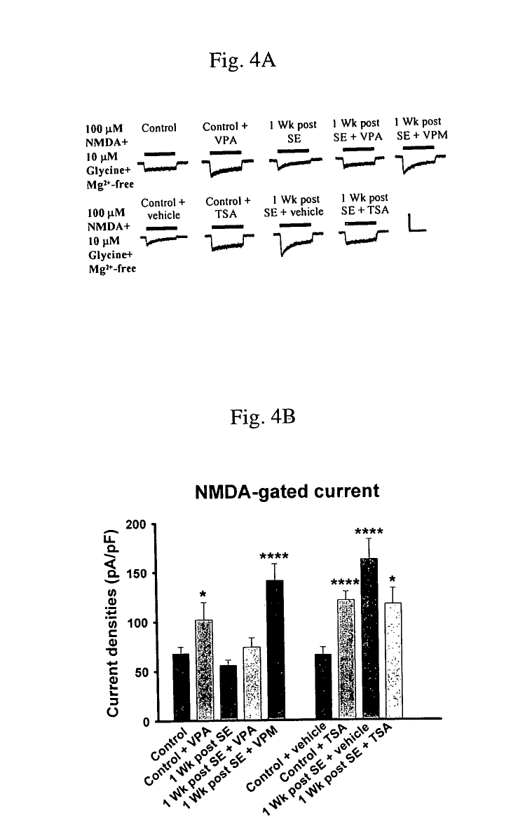

HDAC inhibitors altered NMDA receptor function as shown in FIG. 4. Representative traces depicting DGC responses to NMDA (100 μM) in cells as indicated above are shown in FIG. 4a. As observed in the regulation of AMPAR function, both HDAC inhibitors, VPA and TSA significantly increased DGC NMDAR current densities by 49.9% and 83.1% respectively (“Control”: 68.1±6.7 pA/pF; n=63 cells vs. “Control+VPA”: 102.1±17.5 pA/pF; n=25 cells & “Control+vehicle”: 66.1±7.6 pA/pF; n=22 cells vs. “Control+TSA”: 121.0±9.0 pA/pF; n=39 cells) indicating that there is a tonic HDAC inhibitory activity in regulating NMDAR function in DGCs. In the VPA study, 1 Wk post SE, NMDAR current densities were not significantly altered {Control: 68.1±6.7 pA/pF (n=63 cells) vs. 1 Wk post SE: 55.7±5.6 pA/pF (n=44 cells). However, the ability of VPA to increase NMDAR function observed in DGCs of “Control+VPA” were no longer observed in DGCs of “1 Wk post SE+VPA”. In contrast, VPM was still able to increase NMDAR current densities observed 1 Wk post SE to 141.1±17.3 pA/pF (n=28 cells). In contrast to no significant alteration in NMDAR current densities in the VPA study, a 146.3% increase in NMDAR current densities were seen in DGCs isolated from “1 Wk post SE+vehicle” rats indicating a surgical effect acting on NMDAR {“Control+vehicle”: 66.1±7.6 pA/pF (n=22 cells) vs. “1 Wk post SE+vehicle”: 162.8±20.4 pA/pF (n=19 cells)}. In contrast to the findings in VPA treatment, NMDAR current densities for the group of “1 Wk post SE+TSA” were able to maintain the level similar to the group of “Control+TSA” {“Control+TSA”: 121.0±9.0 pA/pF (n=39 cells) vs. “1 Wk post SE+TSA”: 117.3±16.0 (n=31 cells)}. See FIG. 4b.

Tables 4 and 5 summarize these results below.

TABLE 4
The effects of VPA and VPM

| | Current density | | Desensitization tau | Charge transfer density |
|---|---|---|---|---|
| | Pa/pF | Desensitization % | Msec | (msec) × (nA)/(pF) |
| GABAR | | | | |
| Control | 237.5 ± 12.0 (n = 67) | 84.0 ± 1.6 (n = 74) | 630.4 ± 40.6 (n = 74) | 173.1 ± 13.0 (n = 73) |
| Control + VPA | 243.0 ± 19.3 (n = 22) | 70.3 ± 4.2 (n = 22)*** | 1144.1 ± 146.6 (n = 22)**** | 239.8 ± 27.8 (n = 22)* |
| 1 Wk post SE | 134.3 ± 14.5 (n = 77)**** | 89.1 ± 1.7 (n = 55)* | 491.8 ± 50.1 (n = 55)* | 112.0 ± 18.8 (n = 55)** |
| 1 Wk post SE + VPA | 234.6 ± 12.7 (n = 74) | 76.5 ± 2.1 (n = 73)** | 899.0 ± 90.2 (n = 73)** | 217.0 ± 16.4 (n = 73)* |
| 1 Wk post SE + VPM | 218.6 ± 33.2 (n = 30) | 77.3 ± 2.7 (n = 27)* | 953.3 ± 132.0 (n = 26)** | 249.5 ± 41.5 (n = 26)* |
| AMPAR | | | | |
| Control | 325.4 ± 24.8 (n = 38) | NA | NA | 653.1 ± 52.1 (n = 38) |
| Control + VPA | 385.7 ± 26.4 (n = 26) | NA | NA | 780.4 ± 53.3 (n = 26) |
| 1 Wk post SE | 158.1 ± 16.1 (n = 32)**** | NA | NA | 337.2 ± 31.3 (n = 35)**** |
| 1 Wk post SE + VPA | 228.6 ± 14.5 (n = 66)*** | NA | NA | 459.8 ± 30.8 (n = 66)** |
| 1 Wk post SE + VPM | 302.5 ± 20.2 (n = 27) | NA | NA | 615.3 ± 42.3 (n = 27) |
| NMDAR | | | | |
| Control | 68.1 ± 6.7 (n = 63) | 35.8 ± 2.4 (n = 69) | 1299.3 ± 130.7 (n = 56) | 93.1 ± 7.9 (n = 67) |
| Control + VPA | 102.1 ± 17.5 (n = 25)* | 35.6 ± 4.6 (n = 25) | 669.8 ± 77.3 (n = 21)** | 123.2 ± 18.0 (n = 25) |
| 1 Wk post SE | 55.7 ± 5.6 (n = 44) | 61.9 ± 2.3 (n = 49)**** | 713.1 ± 50.9 (n = 48)*** | 51.1 ± 6.4 (n = 48)*** |
| 1 Wk post SE + VPA | 74.0 ± 9.5 (n = 62) | 43.9 ± 2.7 (n = 61)* | 768.6 ± 51.1 (n = 51)*** | 98.1 ± 12.6 (n = 60) |
| 1 Wk post SE + VPM | 141.1 ± 17.3 (n = 28)**** | 46.7 ± 4.5 (n = 25)* | 787.8 ± 131.5 (n = 23)* | 180.0 ± 22.4 (n = 26)**** |

TABLE 5
The effects of TSA

| | Current density | | Desensitization tau | Charge transfer density |
|---|---|---|---|---|
| | pA/pF | Desensitization % | msec | (msec) × (nA)/(pF) |
| GABAR | | | | |
| Control + vehicle | 209.1 ± 12.0 (n = 15) | 90.4 ± 1.3 (n = 22) | 467.6 ± 64.7 (n = 22) | 114.6 ± 13.4 (n = 16) |
| Control + TSA | 217.9 ± 12.8 (n = 36) | 83.8 ± 1.5 (n = 36)** | 800.7 ± 69.8 (n = 37)** | 169.7 ± 14.2 (n = 37)* |
| 1 Wk post SE + vehicle | 134.1 ± 31.1 (n = 17)* | 85.9 ± 2.6 (n = 18) | 536.5 ± 121.8 (n = 19) | 116.1 ± 34.7 (n = 18) |
| 1 Wk post SE + TSA | 225.6 ± 17.3 (n = 32) | 83.1 ± 2.0 (n = 30)** | 775.7 ± 81.4 (n = 29)** | 185.9 ± 18.4 (n = 30)* |
| AMPAR | | | | |
| Control + vehicle | 306.3 ± 17.0 (n = 23) | NA | NA | 622.6 ± 36.0 (n = 23) |
| Control + TSA | 440.6 ± 26.6 (n = 41)*** | NA | NA | 907.6 ± 57.7 (n = 41)*** |
| 1 Wk post SE + vehicle | 227.2 ± 28.4 (n = 18)* | NA | NA | 449.3 ± 56.4 (n = 18)** |
| 1 Wk post SE + TSA | 343.4 ± 23.5 (n = 33) | NA | NA | 703.3 ± 49.9 (n = 33) |
| NMDAR | | | | |
| Control + vehicle | 66.1 ± 7.6 (n = 22) | 42.4 ± 4.0 (n = 22) | 610.0 ± 53.6 (n = 19) | 88.2 ± 11.8 (n = 21) |
| Control + TSA | 121.0 ± 9.0 (n = 39)**** | 27.1 ± 2.2 (n = 39)*** | 1262.6 ± 197.5 (n = 37)* | 196.2 ± 14.4 (n = 39)**** |
| 1 Wk post SE + vehicle | 162.8 ± 20.4 (n = 19)**** | 56.5 ± 5.2 (n = 17)* | 663.3 ± 160.3 (n = 15) | 185.1 ± 28.0 (n = 19)** |
| 1 Wk post SE + TSA | 117.3 ± 16.0 (n = 31)* | 38.5 ± 3.5 (n = 31) | 1222.1 ± 173.8 (n = 28)** | 153.7 ± 16.6 (n = 31)** |

TSA treatment following SE reduced the subsequent spontaneous seizure frequency as shown in FIG. 5. TSA or vehicle (saline) were injected intraventricularly three times into rats; 2 hr, 6 hr and 24 hr post SE. Rats were subsequently monitored for seizure occurrence and frequency using a video camera for the next 7 weeks. The seizure frequency was reduced in the TSA treated group compared to those of vehicle treatment as indicated in a weekly basis (upper panel; FIG. 5a). The average frequency over the seven weeks monitoring period was reduced more than 70% (lower panel; FIG. 5b). Of additional note, this seizure frequency monitoring was done in animals which were TSA treated following SE, which should provide additional clinical relevance for therapeutic intervention purposes.

FIG. 6 demonstrates that a 1-hr post SE TSA i.p. injection prevented the repression in neurotransmitter receptor-evoked function observed in 1 Wk post SE DGCs. The current density was recovered to the control level. {Control: 237.5±12.0 pA/pF vs. 1 Wk post SE+TSA-i.p.: 235.1±62.3 pA/pF; p>0.9}. Additionally, TSA treatment also prevented part of AMPA receptor current reduction post SE {kainate (KA) along with cyclothiazide, an AMPA receptor-specific desensitization inhibitor application} {Control: 325.4±24.8 pA/pF vs. 1 Wk post SE+TSA-i.p.: 213.7.1±23.3 pA/pF; p<0.05}. In contrast, there was no significant effect for i.p. TSA treatment on NMDAR current density.

### EXAMPLE II

This example described additional experiments related to dosage determination of HDAC inhibitors.

Using Westerns and commercially available antibodies to acetylated histones H3 and H4, hippocampal and neocortical tissue are collected 4 h, 12 h, 1 and 3 days following ICV administration of trichostatin A (see methods, above), and at the same time points following acute IP or continual oral administration of VPA (200 mg/kg SC, or 600 mg/kg/day for 5 days, in water) or sodium butyrate (500 mg/kg SC, or 1-3 g/kg/day for 5 days, in food; Egorin et al. 1999). Levels of anti-acetyl H3 and H4 signal are compared to hippocampal and neocortical tissue in animals receiving a sham ICV injection of PBS, or to untreated controls.

It is anticipated that oral administration of either VPA or sodium butyrate will elevate histone acetylation levels in brain. This has been demonstrated previously (Phiel et al. 2001; Ryu et al. 2003). This example allows quantification of the duration of drug activity and effective dosing to achieve this effect.

### EXAMPLE III

The effects of the HDAC inhibitors, trichostatin A, VPA, and sodium butyrate, on neurotransmitter receptor function and subunit expression levels in DGCs post-SE can be examined as follows. The actual dosage and time of administration of HDAC inhibitors can be determined in accordance with Example IV above.

HDAC inhibitors are administered 1 hr prior to SE, during SE (30 min into SE), and 2 hrs post-SE in effective concentrations. For VPA and sodium butyrate, early acute IP administration of drug as a loading dose is followed by chronic oral administration for 7 days in an additional set of animals.

At varying time points following SE (1, 7, and 14 d), animals are euthanized (4 at each time point and drug treatment regimen), hippocampal slices prepared, and DGCs isolated for recording. Responses to application of GABA, and kainate+cyclothiazide are assessed using patch recording techniques (see FIG. 1). Following recording, the cell cytoplasm is aspirated into the recording electrode, reverse transcribed, and mRNA expression profiling conducted using the Eberwine technique (e.g. Brooks-Kayal et al. 1998; 1999). The usual yield of DGCs from a given isolation exceeds 10 cells recorded, 90% of which successfully profile using the Eberwine method. Therefore, sampling >10 cells from 4 animals constitutes an adequate sample for statistical comparisons. As a control to ensure that HDAC activity has in fact been inhibited by trichostatin A, VPA, or butyrate, cortical tissue is also harvested at the time of dissection in each experiment, and the degree of HDAC blockade assessed using a Western analysis of acetylated histone levels, identical to that described above. Responses are compared to subconvulsive dose pilocarpine animals receiving identical HDAC inhibitor treatments (non-epileptic animals), and to SE animals receiving sham HDAC inhibitor treatments (vehicle alone).

Administration of doses of either VPA, sodium butyrate, or trichostatin A which are effective in altered brain histone acetylation will prevent or blunt the SE-induced down regulation of neurotransmitter receptor expression and function in DGCs, as evidenced by the data presented in FIGS. 1-6.

### EXAMPLE IV

Drugs that prevent or retard the epileptogenic process in injury-induced epilepsies will be of tremendous clinical importance. They will be useful both as a prophylactic treatment in individuals who have received an injury which predisposes them to epilepsy, and as agents administered to patients with ongoing epilepsy, to block the progressive aspects of the disease. Prior to the instant discovery, no such drugs were known to exist.

There are currently several animal models of TLE which exhibit many of the primary clinical correlates of this disease, and also result in a condition characterized by recurrent spontaneous seizures, which develops following a latent period with no overt seizures. Both of these aspects of an acquired epilepsy model (spontaneous seizures, and a latent period) are absolute prerequisites for antiepileptogenic drug testing. In this example, the rat pilocarpine model of SE-induced TLE is used to screen candidate compounds for efficacy as antiepileptogenic drugs.

HDAC inhibitor treatments based on the Examples above provide optimal dosing strategies to block SE-induced transcriptional repression. Drugs tested are VPA, sodium butyrate, and trichostatin A. VPA and sodium butyrate are administered orally. SC booster doses are administered during and immediately following SE, because animals do not eat normally when seizing, or when post-ictal. The post-ictal lethargic period lasts for about 1 day post SE. Trichostatin A is administered ICV but may also be administered IP. Details of treatment regimens of all 3 drugs are described above. Due to the variable onset of SE in the pilocarpine model, 10-12 animals are monitored for each treatment regimen. Both treated (SE and histone deacetylase treatment) and control (SE without drug treatment and no SE with drug treatment) animals are placed in clear sided cages, which are stacked 3×2 in the field of a video camera. A digital camera is also contained within the field. Animals are coded so observers do not know the state of the animals, and the order of the animals in the cage array is changed daily. Animals are taped 24 hrs/day beginning immediately after cessation of drug treatment. Virtually all (>95%) control (VPA untreated) pilocarpine animals become epileptic with 4-6 weeks following SE, so monitoring is ceased at 6 weeks. Following taping, videos are reviewed by expert observers blind to the animal treatment group. Spontaneous Class 3 or higher (convulsive) seizures on the Racine scale are counted for each animal, and seizure frequency and severity determined, as these indices vary with treatment and time post-SE. After the 6 week monitoring period, animals are perfused, and a hippocampal pathology workup conducted, examining cell loss with Cresyl violet staining, mossy fiber sprouting with Timm's stain, and parvalbumin staining as an index of interneuron loss.

For further monitoring, bilateral depth electrodes and in some cases trichostatin A cannulas are implanted, and animals are allowed to heal for 10 days following surgery. The commutators allow for 4 depth electrodes, so bilateral hippocampal electrodes, and bilateral amygdala electodes are implanted. Trichostatin animals also have cannulas implanted. Animals (n=5 for each group) receive an optimal dosing strategy for each drug (VPA, butyrate, and trichostatin A). Pilocarpine is administered according to the usual protocol (see Methods, above). Animals are then connected to the commutator setup, and recorded continually in an undisturbed manner for the next 6 weeks, during SE and in the post-SE period. EEG is amplified and recorded on the Nicolet digital video/EEG amplifier/computer setup, and stored on DVDs for later analysis. Video recordings of animal behavior are also stored. The frequency and complexity of interictal bursts are analyzed, as are seizure frequency, duration, and behavioral responses, as these measures vary with time post-SE and drug treatment. Responses are compared to animals receiving pilocarpine with sham oral dosing, or following a sham 5 ul PBS intraventricular infusion.

### EXAMPLE V

This example demonstrates that brain injuries other than status epilepticus produce neurotransmitter receptor transcription repression.

Methods are described in Coulter et al. (1996). Briefly, anesthetized animals are implanted with a Luer-loc syringe hub, at −3 mm from Bregma, 3 mm lateral to the sagital suture. A day later, animals are reanesthetized, and attached to a fluid percussion injury apparatus (Dixon et al. 1988). A pendulum weight is released from sufficient height to provide a moderate to severe FPI, usually 2.0-2.2 atm measured at the pressure transducer built into the device. Animals remain unresponsive following injury for a minimum of 30 sec, assessed by hindlimb pinch.

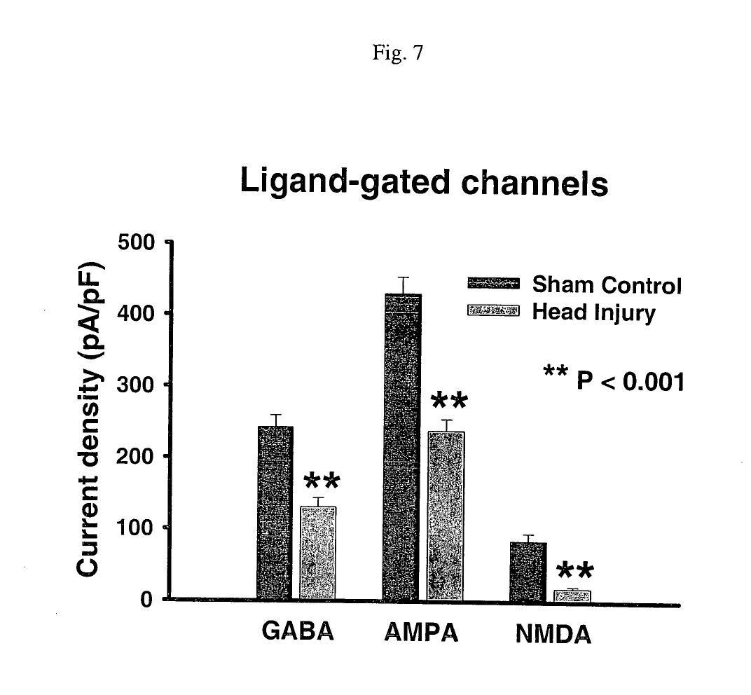

Neurons had significantly smaller GABA, AMPA, and NMDA currents post-head injury, indicating that transcriptional repression of neurotransmitter receptor subunits also occurs following an alternate form of head injury (see FIG. 7).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

### Literature Cited

Andre V, Ferrandon A, Marescaux C, Nehlig A. The lesional and epileptogenic consequences of lithium-pilocarpine-induced status epilepticus are affected by previous exposure to isolated seizures: effects of amygdala kindling and maximal electroshocks. Neuroscience 2000;99(3):469-81
Babb, T. L., Brown, W. J., Pretorius, J., Davenport, C., Lieb, J. P., and Crandall, P. H. Temporal lobe volumetric cell densitites in temporal lobe epilepsy. Epilepsia 25: 729-740, 1984.
Babb, T. L., Kupfer, W. R., Pretorius, J. K., Crandall, P. H., and Levesque, M. F. Synaptic reorganization by mossy fibers in human epileptic fascia dentata. Neuroscience 42: 351-363, 1991.
Ballas, N., Battaglioli, E., Atouf, F., Andres, M. E., Chenoweth, J., Anderson, M. E. et al. Regulation of neuronal traits by a novel transcriptional complex. Neuron 31: 353-365, 2001.
Baylin, S. B. and Herman, J. G. DNA hypermethylation in tumorigenesis. TIC 16: 168-174, 2000.
Blaheta, R. A. Cinatl, J. Jr. Anti-tumor mechanisms of valproate: a novel drug role for an old drug. Medicinal Research Reviews 22: 492-511, 2002.
Brooks-Kayal, A., Shumate, M. D., Hong, Y., Rikhter, T. Y., and Coulter, D. A. Selective changes in single cell GABA_{A} receptor subunit expression correlate with altered function in epileptic hippocampus. Nature (Med) 4 (10): 1166-1172, 1998.
Brooks-Kayal, A. R., Shumate, M. D., Jin, H., Lin, D. D., Rikhter, T. Y., Holloway, K., and Coulter, D. A. Human neuronal-aminobutyric acidA receptors: Coordinated subunit mRNA expression and functional correlates in individual dentate granule cells. J. Neurosci. 19: 8312-8318, 1999.
Brooks-Kayal, A. R., Shumate, M. D., Jin, H., Rikhter, T. Y., Kelly, M. E., and Coulter, D. A. GABA_{A} receptor subunit expression predicts functional changes in dentate granule cells during postnatal development. J. Neurochem 77: 1266-1278, 2001.
Buhl, E. H., Otis, T. S., and Mody, I. Zinc-induced collapse of augmented inhibition by GABA in a temporal lobe epilepsy model. Science 271: 369-372, 1996.
Calderone, A., Jover, T., Noh, K.-M., Tanaka, H., Yokata, H., Lin, Y., Grooms, S. Y., Regis, R., Bennett, M. V. L., and Zukin, R. S. Ischemic insults derepress the gene silencer REST in neurons destined to die. J. Neurosci. 23(6): 2112-2121, 2003.
Chang J G, Hsieh-Li H M, Jong Y J, Wang N M, Tsai C H, Li H. Treatment of spinal muscular atrophy by sodium butyrate. Proc Natl Acad Sci USA. 2001 Aug. 14;98(17):9808-13.
Chang, J. C., et al. Inhibition of DNA methylation and reactivation of silenced genes by Zebularine. J. Nat. Canc. Inst. 95: 399-409, 2003.
Chawla, S., Vanhoutte, P., Arnold, F. J. L., Huang, C. L.-H., and Bading, H. Neuronal activity-dependent nucleocytoplasmic shuttling of HDAC4 and HDAC5. J. Neurochem. 85: 151-159, 2003.
Cohen, A. S., Lin, D. D., Quirk, G. L. and Coulter, D. A. Dentate granule cell GABAA receptors in epileptic hippocampus: enhanced synaptic efficacy and altered pharmacology. European Journal of Neuroscience 17:1607-1616, 2003.
Coulter, D. A., Rafiq, A., Shumate, M., Gong, Q.-Z., DeLorenzo, R. J., and Lyeth, B. G. Brain injury-induced enhanced limbic epileptogenesis: Physiological and anatomical parallels to an animal model of temporal lobe epilepsy. Epilepsy Research 26: 81-91, 1996.
De Ruijter, A. J. M., van Gennip, A. H., Caron, H. N., Kemp, S., and van Kuilenburg, A. B. P. Histone deacetylases (HDACs): characterization of the classical HDAC family. Biochem. J. 370: 737-749, 2003.
De Lanerolle, N. C., Kim, J. H., Robbins, R. J., and Spencer, D. D. Hippocampal interneuron loss and plasticity in human temporal lobe epilepsy. Brain Res. 495: 387-395, 1989.
Dixon, C. E., Lyeth, B. G., Povlishock, J. T., Findling, R. L., Hamm, R. J., Marmarou, A., Young, H. F., and Hayes, R. L. A fluid percussion model of experimental brain injury in the rat. J. Neurotrauma 5: 91-104, 1988.
Egorin, M. J., Yuan, Z.-M., Sentz, D. L., Plaisance, K., and Eiseman, J. L. Plasma pharmacokinetics of butyrate after intravenous administration of sodium butyrate or oral administration of tributylin or sodium butyrate to mice and rats. Cancer Chemother. Pharmacol. 43: 445-453, 1999.
Gibbs III, J. W., Shumate, M. D., and Coulter, D. A. Differential epilepsy-associated alterations in postsynaptic GABAA receptor function in dentate granule and CA1 neurons. J. Neurophysiol. 77: 1924-1938, 1997.
Goffin, J., and Eisenhauer, E. DNA methyltransferase inhibitors-state of the art. Annals of Oncology 13: 1699-1716, 2002.
Gottlicher, M., Minucci, S., Zhu, P., Kramer, O. H., Schimpf, A., Giavara, S., Sleeman, J. P., Lo Coco, F., Nervi, C., Pelicci, P. G., and Heinzel, T. Valproic acid defines a novel class of HDAC inhibitors inducing differentiation of transformed cells. EMBO J. 20: 6969-6978, 2001.
Griffith, E. C., Cowan, C. W. and Greenberg, M. E. REST acts through multiple deacetylase complexes. Neuron 31: 339-344, 2001.
Hockly E, Richon V M, Woodman B, Smith D L, Zhou X, Rosa E, Sathasivam K, Ghazi-Noori S, Mahal A, Lowden P A, Steffan J S, Marsh J L, Thompson L M, Lewis C M, Marks P A, Bates G P. Suberoylanilide hydroxamic acid, a histone deacetylase inhibitor, ameliorates motor deficits in a mouse model of Huntington's disease. Proc Natl Acad Sci USA 2003 Feb. 18;100(4):2041-6
Houser, C. R., et al. Altered patterns of dynorphin immunoreactivity suggest mossy fiber reorganization in human hippocampal epilepsy. J. Neurosci. 10: 267-282, 1990.
Hsu, F.-C., and Coulter, D. A. Differential alterations in GABA_{A}, receptor function in dentate granule and CA1 neurons during the latent period of temporal lobe epilepsy. Soc. Neurosci. Abstr. 28, 599.8, 2002.
Hsu, F. C., Rikhter, T. Y., Ahmed, H., and Coulter, D. A. Selective Maintenance of NMDA Receptor Function in Dentate Granule Cells During the Latent Period of Temporal Lobe Epilepsy. Soc. Neurosci. Abstr., 27 558.3, 2001.
Huang, Y., Myers, S. J., and Dingledine, R. Transcriptional repression by REST: recruitment of Sin3A and histone deacetylase to neuronal genes. Nature Neuroscience 2: 867-872, 1999.
Huang, Y., Doherty, J. J., and Dingledine, R. Altered histone acetylation at glutamate receptor 2 and brain-derived neurotrophic factor genes is an early event triggered by status epilepticus. J. Neuroscience 22: 8422-8428, 2002.
Jones, P. A., and Baylin, S. B. The fundamental role of epigenetic events in cancer. Nature Reviews in Genetics 3: 415-428, 2002.
Johnstone, R. W. Histone-deacetylase inhibitors: novel drugs for the treatment of cancer. Nature Reviews 1: 287-299, 2002.
Karpf, A. R., and Jones, D. A. Reactivating the expression of methylation silenced genes in human cancer. Oncogene 21: 5496-5503, 2002.
Kelly M. E., Rikhter T., Luscher B., and Coulter, D. A., (2000). Altered Transcriptional Regulation of GABA_{A} Receptor Expression Following Status Epilepticus (SE): Implications for Epileptogenesis. Epilepsia 41, S(4), p. 21.
Li, J., Liu, Q., Wang, W., Wade, P., and Wong, J. Specific targeting and constitutive association of histone deacetylase complexes during transcriptional repression. Genes and Development 16: 687-692, 2002.
Longo B M, Mello L E. Supragranular mossy fiber sprouting is not necessary for spontaneous seizures in the intrahippocampal kainate model of epilepsy in the rat. Epilepsy Res 1998 September; 32(1-2):172-82
Loscher W. Valproate: a reappraisal of its pharmacodynamic properties and mechanisms of action. Prog Neurobiol 1999 May; 58(1):31-59
Lunyak, V. V., Burgess, R., Prefontaine, G. G., Nelson, C., Sze, S.-H., Chenoweth, J., Schwartz, P., Pevzner, P. A., Glass, C., Mandel, G., and Rosenfeld., M. G. Corepressor-dependent silencing of chromosomal regions encoding neuronal genes. Science 298: 1747-1752, 2002.
Mouritzen Dam, A. Epilepsy and neuron loss in the hippocampus. Epilepsia 21: 617-629, 1980.
Naruse Y, Aoki T, Kojima T, Mori N. Neural restrictive silencer factor recruits mSin3 and histone deacetylase complex to repress neuron-specific target genes. Proc Natl Acad Sci USA 1999 Nov. 23;96(24):13691-6
Nusser, Z., .Hajos, N., Somogyi, P., and Mody, I. Increased number of synaptic GABAA receptors underlies potentiation at hippocampal inhibitory synapses. Nature 395: 172-177, 1998.
Otis, T. S., De Koninck, Y., and Mody, I. Lasting potentiation of inhibition is associated with an increased number of -aminobutyric acid type A receptors activated during miniature inhibitory postsynaptic currents. Proc. Natl. Acad. Sci. USA 91: 7698-7702, 1994.
Palm, K.,. Belluardo, N., Metsis, M., and Timmusk, T. Neuronal expression of zinc finger transcription factor REST/NRSF/XBR gene. J. Neuroscience 18: 1280-1296, 1998.
Phiel, C. J., Zhang, F., Huang, E. Y., Guenther, M. G., Lazar, M. A., and Klein, P. S. Histone deacetylase is a direct target of valproic acid, a potent anticonvulsant, mood stabilizer, and teratogen. J. Biol. Chem. 276 (39): 36734-36741, 2001.
Rikhter, T. Y., Shumate, M. D., Brooks-Kayal, A. R., Lynch, D. R., and Coulter, D. A. Altered GABA_{A} and NMDA receptor subunit mRNA expression in single dentate granule cells of pilocarpine treated rats before the onset of epilepsy. Soc Neuroscience Abstracts 25, 842, 1999.
Rikhter, T. Y., Shumate, M. D., Brooks-Kayal, A. R., Jin, H., Lynch, D. R., and Coulter, D. A. Altered GABA_{A}, AMPA, KA, and NMDA receptor subunit mRNA expression in single dentate granule cells (DGCs) of pilocarpine (PILO) treated rats before the onset of epilepsy. Soc Neuroscience Abstracts 26: 1656, 2000.
Ryu, H., et al. Histone deacetylase inhibitors prevent oxidative neuronal death independent of expanded polyglutamine repeats via an Sp-1-dependent pathway. PNAS 100: 4281-4286, 2003.
Shumate, M. D., Lin, D. D., Gibbs III, J. W., Holloway, K. L., and Coulter, D. A. Physiological properties of GABA_{A} receptors in epileptic human dentate granule cells: Comparison to epileptic and control rat. Epilepsy Res. 32: 114-28, 1998.
Sutula, T., Cascino, G., Cavazos, J., Parada, I., and Ramirez, L. Mossy fiber synaptic reorganization in the epileptic human temporal lobe. Ann. Neurol. 26: 321-330, 1989.
Szyf, M. Towards a pharmacology of DNA methylation. TIPS 22: 350-354, 2001.
Tauck, D. L. and Nadler, J. V. Evidence of functional mossy fiber sprouting in hippocampal formation of kainic acid-treated rats. J. Neurosci. 5: 1016-1022, 1985.
Thiagalingam, S., Cheng, K-H., Lee, H.-J., Mineva, N., Thiagalingam, A., and Ponte, J. F. Histone deacetylases: unique players in shaping the epigenetic histone code. Ann. N. Y. Acad. Sci. 983: 84-100, 2003.
Tsunashima, K., et al. GABA_{A} receptor subunits in the rat hippocampus III: altered messenger RNA expression in kainic acid-induced epilepsy. Neuroscience 80: 1019-1032, 1997.
Zhang X, Cui S S, Wallace A E, Hannesson D K, Schmued L C, Saucier D M, Honer W G, Corcoran M E. Relations between brain pathology and temporal lobe epilepsy. J Neurosci 2002 Jul. 15;22(14):6052-61.

### REFERENCES DESCRIBED IN TABLES 1, 2, and 3

1. Ailenberg, M. and Silverman, M. (2002) Trichostatin A-histone deacetylase inhibitor with clinical therapeutic potential-is also a selective and potent inhibitor of gelatinase A expression. Biochem. & Biophysic. Res. Commu. 298: 110-115.
2. Boffa, L. C., Vidali, G., Mann, R. S. and Allfrey, V. G. (1978) Suppression of histone deacetylation in vivo and in vitro by sodium butyrate. J. Biol. Chem. 253: 3364-3366.
3. Demary, K., Wong, L. and Spanjaard, R. A. (2001) Effects of retinoic acid and sodium butyrate on gene expression, histone acetylation and inhibition of proliferation of melanoma cells. Cancer Lett. 163:103-107.
4. Eyal, S., Yagen, B., Sobol, E., Altschuler, Y., Shmuel, M. and Bialer, M. (2004) The activity of antiepileptic drugs as histone deacetylase inhibitors 45: 737-744.
5. Göttlicher, M., Minucci, S., Zhu, P., Krämer, O. H., Schimpf, A., Giavara, S., Sleeman, J. P., Coco, F. L., Nervi, C., Pelicci, P. G. and Heinzel, T. (2001) Valproic acid defines a novel class of HDAC inhibitors inducing differentiation of transformed cells. EMBO J. 20: 6969-6978.
6. Gurvich, N., Tsygankova, O. M., Meinkoth, J. L. and Klein, P. S. (2004) Histone deacetylase is a target of valproic acid-mediated cellular differentiation. Cancer Res. 64: 1079-1086.
7. Huang, Y., Doherty, J. J. and Dingledine, R. (2002) Altered histone acetylation at glutamate receptor 2 and brain-derived neurotrophic factor genes is an early event triggered by status epilepticus. J. Neurosci. 22: 8422-8428.
8. Jeong, M.-R., Hashimoto, R., Senatorov, V. V., Fujimaki, K., Ren, M., Lee, M. S. and Chuang, D.-M. (2003) Valproic acid, a mood stabilizer and anticonvulsant, protects rat cerebral cortical neurons from spontaneous cell death: a role of histone deacetylase inhibition. 542: 74-78.
9. Phiel C. J., Zhang, F., Huang, E. Y., Guenther, M. G., Lazar, M. A. and Klein P. S. (2001) Histone deacetylase is a direct target of valproic acid, a potent anticonvulsant, mood stabilizer, and teratogen. J. Biol. Chem. 276: 36734-36741.
10. Ren, M.-R, Leng, Y., Jeong, M., Leeds, P. R. and Chuang, D.-M. (2004) Valproic acid reduces brain damage induced by transient focal cerebral ischemia in rats: potential roles of histone deacetylase inhibition and heart shock protein induction. J. Neurochem. 89: 1358-1367.

1. A method for preventing or inhibiting the onset of epilepsy in a patient in need thereof comprising administering an effective amount of a transcriptional derepressor to said patient within a critical window of a brain injury, thereby inhibiting onset of epilepsy.

2. The method of claim 1, wherein said transcriptional derepressor is at least one HDAC inhibitor.

3. The method of claim 2, wherein said HDAC inhibitor is selected from the group consisting of valproic acid (VPA), trichostatin A, suberoyl anilide bishydroxamide (SAHA), m-carboxycinnamic acid (CBHA), oxamflatin, trapoxin A, topiramate, levitiracetam, apicidin, and sodium butyrate.

4. The method of claim 1, wherein said critical window is selected from the group consisting of before anticipated brain injury, within 24 hours after brain injury, 24 hours to 7 days after injury, 7-10 days after injury, 24 hours to 2 weeks after injury, 24 hours to two months after injury, and 24 hours to 1 year after injury.

5. The method of claim 1, wherein said brain injury is caused by a condition selected from the group consisting of status epilepticus, trauma such as concussive injuries or penetrating head wounds, brain tumor, alcoholism, Alzheimer's disease, stroke, heart attack, meningitis, AIDS, viral encephalitis, and hydrocephalus.

6. The method of claim 1, wherein an additional anti-convulsant is administered in combination with said transcriptional derepressor.

7. The method of claim 1, wherein said transcriptional derepressor is administered by a method selected from the group consisting of oral, intravenous, cutaneous or subcutaneous, nasal, intramuscular, intraperitoneal, and intracerebroventricular routes.

8. The method of claim 1, wherein said transcriptional derepressor is administered to an adult at a dosage of about 100 to 500 mg/kg/day.

9. The method of claim 1, wherein said transcriptional derepressor is administered to a child at a dosage of about 50 to 500 mg/kg/day.

10. The method of claim 1, wherein said transcriptional derepressor targets transcriptional repression of a neurotransmitter receptor selected from the group consisting of GABA_{A} receptors α1-6, β1-3, γ1-3, δ, ε, π; AMPA receptor GluR1-4; NMDA receptor, kainate receptors GluR5-7, and KA1-2.
